(19) United States
(12) Patent Application Publication
(10) Pub. No.: US 2010/0330136 A1
(43) Pub. Date: Dec. 30, 2010

(54) USE OF CATIONIC SURFACTANTS FOR THE PROTECTION AGANST TOOTH EROSION

(76) Inventor: Xavier Rocabayera Bonvila, Palau-Solita (ES)

Correspondence Address: RANKIN, HILL & CLARK LLP, 23755 Lorain Road - Suite 200, North Olmsted, OH, 44070-2224, US

(21) Appl. No.: 12/812,813

(22) PCT Filed: Feb. 11, 2009
(86) PCT No.: PCT/EP09/51587

§ 371 Date: Aug. 11, 2010

Related U.S. Application Data
(60) Provisional application No. 61/045,393, filed on Apr. 16, 2008.

(30) Foreign Application Priority Data
Feb. 13, 2008 (EP) 08382007.6

(51) Int. Cl. A61K 8/72; A61K 8/44; A61K 8/02; C07C 279/14; A61K 8/81; A61K 8/85; A61K 8/11; A61K 8/92; A61Q 11/00

(52) U.S. Cl. 424/401; 424/54; 424/48; 560/168

(57) ABSTRACT

This invention relates to a use of a composition for protecting oral cavity and teeth containing ethyl-Nα-lauroyl-L-arginate HCl (LAE), a corresponding composition and a corresponding method for protecting teeth. Because of nearly epidemic incidence of dental erosion, there is a continuing need for improved products that provide protection against dental erosion. Hence, it is an object of the present invention to protect teeth against dental erosion. Surprisingly, it has been found that ethyl-N^{α}-lauroyl-L-arginate HCl (LAE) and its salts attach to teeth and provide protection of teeth from erosion especially caused by the action of acid in food products and drinks such as chewing gum and lozenges compositions. Special advantages of this invention are: (a) sustained coating of the teeth by LAE and its homologues; (b) the coating agent provides a source of neutralizing plaque acids by providing sustained base formation. This is because arginines in LAE is degraded by bacteria on tooth in the mouth to produce ammonia. Thus, this chemistry not only provides the coating protection but also generates base to maintain pH balance on a sustained basis; and (c) avoid the use of brushing with abrasive cleansers.

### FIELD OF THE INVENTION

This invention relates to the use of cationic surfactants with antimicrobial properties for protecting teeth and the oral cavity.

### BACKGROUND ART

Tooth erosion, or tooth wear, is the loss of tooth structure. Basically, tooth erosion refers to the wearing away of the hard part of the teeth, the so-called enamel. Tooth erosion is caused by the consumption of carbonated beverages, fruit juice and highly acidic foods. The acid in food and drinks can cause tooth enamel to wear away, leading to the erosion of the tooth (sensitivity, discoloration, rounded teeth, cracks, severe sensitivity or cupping).

Other aspects which may cause a permanent loss of tooth mineral (tooth erosion) are the action of chemicals, brushing teeth with abrasive cleansers or eating disorders like regurgitation of acid produced in the stomach which thereupon comes in contact with the teeth during the process of vomiting, or reflux.

Usually, the calcium contained in saliva helps the normal process of remineralization of tooth after the consumption of small amounts of acid. However, the presence of a lot of acid in the mouth removes the calcium from the teeth faster than it can be replaced, conducting to the tooth erosion.

The incidence and severity of dental erosion is on the rise with the increase of consumption of acidic beverages and juices. The pH and titratable acidity of acidic beverages have been identified as the main causes in the initiation and progression of dental erosion; the greater the concentration of acid in the beverage the more damaging to teeth it becomes (Lussi, Caries Res. 1995; 29: 349-354).

Thus, methods have been disclosed to modify acidic food and beverages products in order to prevent erosive effect on teeth (U.S. Pat. No. 6,383,473; U.S. Pat. No. 6,319,490). The author of these patents provides a method to reduce tooth erosion by adding a calcium compound to acidic compositions for oral consumption.

There are other prior art technologies that use ingredients in dentifrices to reduce the solubility of enamel. In U.S. Pat. No. 3,914,404 this purpose is reached by the development of novel dentifrices (i.e.: mouth washes, toothpastes, tooth powders and chewing gums) containing tin in stannous form as a substantially water-insoluble, non-ionizing chelate of a synthetic amino carboxylic acid.

Toothpastes by themselves are one of the causes of erosion when they contain abrasive cleansers.

Other methods have focused on protecting tooth from dental plaque formation that it is a byproduct of microbial growth (US 2005/0031551, US 2006/0193791, US 2007/0014740 and US 2007/0140990). The problems associated with the formation of plaque on the teeth are the tendency of the plaque to build up and also to produce gingivitis, periodontitis, dental caries, halitosis and dental calculus. As dental plaque adheres firmly to dental surfaces, it can be removed only with difficulty through a rigorous brushing regiment, which at the end could cause dental erosion itself. The above-cited documents describe oral compositions that reduce or prevent plaque formation using cationic surfactants, wherein one example of it was ethyl lauroyl arginine hydrochloride salt (LAE).

Because of the nearly epidemic incidence of dental erosion, there is a continuing need for improved products that provide protection against dental erosion. Hence, it is an object of the present invention to protect teeth against dental erosion.

The object of the invention is solved by the use of specific cationic surfactants with antimicrobial properties. These products are preferably applied in oral consumption products such as sweets, candies, tablets, lozenges, lollies, chews, jellies, gums, drops and dry powder blends, such as powdered drinks intended for dissolution, for example in the water.

Cationic surfactants are known as preservatives used in the food, cosmetic and pharmaceutical industry. Cationic surfactants have turned out to be highly effective against microbial proliferation and at the same time safe for intake in humans and mammals in general. For all of this, cationic surfactants are an attractive tool in the industry.

It has been demonstrated that cationic surfactants according to formula (1) derived from the condensation of fatty acids and esterified dibasic amino acids are highly effective protective substances against microorganisms.

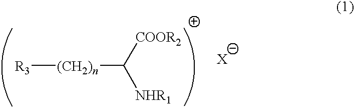

where:
X⁻ is a counter ion derived from an inorganic or organic acid, preferably Br⁻, Cl⁻, or HSO₄⁻
R₁: is a straight alkyl chain of a saturated fatty acid or a hydroxy acid having 8 to 14 carbon atoms linked to the α-amino group via an amide bond,
R₂: is a straight or branched alkyl chain from 1 to 18 carbon atoms or an aromatic group and

### R₃: is:

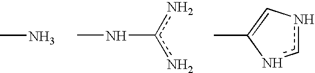

where n is from 0 to 4.

The organic acids which may be the source of the counter ion X⁻ can be citric acid, lactic acid, acetic acid, fumaric acid, maleic acid, gluconic acid, propionic acid, sorbic acid, benzoic acid, carbonic acid, glutamic acid or other amino acids, lauric acid and fatty acids such as oleic acid and linoleic acid, whereas the inorganic acids can be phosphoric acid, nitric acid and thiocyanic acid.

The phenolic compound which may be the basis of the anion X⁻ is for instance butylated hydroxyanisole (BHA) and the related butylated hydroxytoluene, tertiary butyl hydroquinone and parabens such as methylparaben, ethylparaben, propylparaben and butylparaben.

The most preferred compound of the above class of cationic surfactants is the ethyl ester of the lauramide of the arginine monohydrochloride, hereafter referred to as LAE (CAS No. 60372-77-2), this product is now well-known for its use as an antimicrobial agent. In practical use LAE turned out to be well tolerated and to display a very low toxicity to human beings. LAE has the chemical structure of formula (2) displayed hereafter.

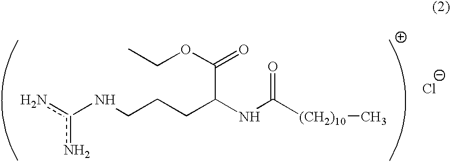

The compound LAE is remarkable for its activity against different microorganisms, like bacteria, moulds and yeasts which can be present in food products (WO 03/034842) and also in cosmetic formulations and preparations (WO 03/013453, WO 03/013454 and WO 03/043593).

The general preparation of the cationic surfactants is described in Spanish patent ES 512643 and international patent applications WO 96/21642, WO 01/94292 and WO 03/064669.

LAE, also known as lauric arginate, is manufactured by Laboratorios Miret, S.A. (LAMIRSA, Spain). Lauric arginate is listed by the FDA (Food and Drug Administration) as being a GRAS substance (Generally Recognized As Safe) under GRN 000164. The USDA (United States Department of Agriculture) has approved its use in meat and poultry products (FSIS Directive 7120.1).

The metabolism of the above cationic surfactant of formula (2) in rats has been studied, these studies have shown a fast absorption and metabolisation into naturally-occurring amino acids and the fatty acid lauric acid, which are eventually excreted as carbon dioxide and urea. Toxicological studies have demonstrated, that LAE is completely harmless to animals and humans.

Therefore, LAE and related compounds are particularly suitable to be used in the preservation of all perishable food products. LAE and related compounds are equally suitable for use in cosmetic products.

As has been previously mentioned, the cationic surfactants are remarkable for their inhibitory action over the proliferation of different microorganisms, such as bacteria, fungi and yeasts. The minimum inhibitory concentrations of LAE are shown in the following table 1:

TABLE 1

| | | M.I.C. |
|---|---|---|
| Kind | Microorganism | (ppm) |
| Gram + | Arthrobacter oxydans ATCC 8010 | 64 |
| Bacteria | Bacillus cereus var mycoide ATCC 11778 | 32 |
| | Bacillus subtilis ATCC 6633 | 16 |
| | Clostridium perfringens ATCC 77454 | 16 |
| | Listeria monocytogenes ATCC 7644 | 10 |
| | Staphylococcus aureus ATCC 6538 | 32 |
| | Micrococcus luteus ATCC 9631 | 128 |
| | Lactobacillus delbrueckii | 16 |
| | ssp lactis CECT 372 | |
| | Leuconostoc mesenteroides CETC 912 | 32 |
| | Streptococcus mutans ATCC 25175 | 64 |
| Gram − | Alcaligenes faecalis ATCC 8750 | 64 |
| Bacteria | Bordetella bronchiseptica ATCC 4617 | 128 |
| | Citrobacter freundii ATCC 22636 | 64 |
| | Enterobacter aerogenes CECT 689 | 32 |
| | Escherichia coli ATCC 8739 | 32 |
| | Escherichia coli 0157H7 | 20 |
| | Klebsiella pneumoniae | 32 |
| | var pneumoniae CECT 178 | |
| | Proteus mirabilis CECT 170 | 32 |
| | Pseudomonas aeruginosa ATCC 9027 | 64 |
| | Salmonella typhimurium ATCC16028 | 32 |
| | Serratia marcenses CECT 274 | 32 |
| | Mycobacterium phlei ATCC 41423 | 2 |
| Fungi | Aspergillus niger ATCC14604 | 32 |
| | Aureobasidium pullulans ATCC 9348 | 16 |
| | Gliocadium virens ATCC 4645 | 32 |
| | Chaetonium globosum ATCC 6205 | 16 |
| | Penicillium chrysogenum CECT 2802 | 128 |
| | Penicillium funiculosum CECT 2914 | 16 |
| Yeast | Candida albicans ATCC 10231 | 16 |
| | Rhodotorula rubra CECT 1158 | 16 |
| | Saccharomyces cerevisiae ATCC 9763 | 32 |

### SUMMARY OF THE INVENTION

Surprisingly, it has been found that the cationic surfactants such as ethyl N^{α}-lauroyl-L-arginate HCl (LAE) and its salts attach to teeth and provide protection of teeth from erosion especially caused by the action of acid in food and drink products such as sweets, candies, tablets, lozenges, lollies, chews, jellies, gums, drops and dry powder blends such as powdered drinks intended for dissolution. The invention provides the formulation of the above mentioned food and drinks products with cationic surfactants and more specifically with LAE. These formulations show surprising results like: (a) sustained coating of the teeth by the cationic surfactants such as LAE and its homologues, which implies an anti-attachment effect against microorganisms responsible to damage teeth, at the same time that there is an antimicrobial effect produced by the presence of the cationic surfactants such as LAE; and (b) the coating agent provides a source of neutralizing plaque acids by providing sustained base formation. This is because arginine in LAE or the corresponding dibasic amino acid in the other cationic surfactants used according to the invention is degraded by bacteria on the teeth in the mouth to produce ammonia. Thus, this chemistry not only provides the coating protection but also generates base to maintain pH balance on a sustained basis; and its use allows to (c) avoid the use of brushing with abrasive cleansers.

### DESCRIPTION OF THE PREFERRED EMBODIMENTS

The special feature and unique aspect of the present invention will become evident to those skilled in the art from the detailed description which follows.

Preferably, the composition is in the form of a chewing gum or a lozenge, but it may also be in the form of other oral consumption products. Chewing gum comprises a gum base matrix as a major component which includes a gum base material which may be selected from the numerous water- and saliva-insoluble gum materials known in the art. Illustrative examples of suitable polymer gum base include both synthetic and natural elastomers and rubbers, as well as mixtures thereof. It is for example possible to use substances of plant origin such as chicle, jelutong, gutta percha and crown gum. Synthetic elastomers such as butadiene-styrene copolymer, isobutylene-isoprene copolymer, polyethylene, polyisobutylene, polyester such as polyvinyl acetate and mixtures of any of the foregoing may be particularly useful.

The preferred embodiment of the chewing gum will provide controlled release of LAE and the flavor ingredients in an oral environment. Some of the known sustained release delivery systems for the control of release of active ingredients over a sustained period include a wax matrix system, the “miniature osmotic pump system” and the Forest Synchron Drug Delivery System.

The wax matrix system provides the active ingredient LAE and flavor dispersed in a wax binder which slowly dissolves in saliva to gradually release LAE and flavors. This system encapsulates the active ingredient in various polymeric coatings, having a varying degree of solubility depending on pH and or enzymes to control the release.

In the “miniature osmotic pump system” an active ingredient is coated with a semi-permeable membrane. The pump works when water soluble LAE is released through a hole drilled into the membrane.

The preferred controlled-release chewing gum and lozenge system is the Forest Synchron Drug Delivery System. In this system the active ingredient LAE is dispersed with flavor uniformly and homogeneously throughout a mass of water-swellable modified cellulosic powder or fibers forming a coherent network as a matrix. The mixture of fibrous or powdery mass and active ingredient LAE, with optional additives such as flavoring, binder, lubricant, and processing aids, is compacted in chewing gum sticks, pellets or lozenges prior to use. This delivery system, when exposed to saliva environment, releases active ingredient, LAE, in the mouth for coating the teeth and providing protection against tooth erosion. Further details of the Forest Synchron Delivery System are disclosed in U.S. Pat. No. 3,870,790; U.S. Pat. No. 4,226,849; U.S. Pat. No. 4,357,469 assigned to Forest Laboratories and are incorporated herein.

The gum base matrix may additionally contain other ingredients well-known in the art which are selected from the group consisting of plasticizers and softeners to help reduce the viscosity of the gum base to a desirable consistency and to improve the overall texture and bite. These compounds are also noted for their emulsifying properties. As non-limiting examples, compounds such as lecithin, mono- and di-glycerides, lanolin, stearic acid, potassium stearate, glycerol triacetate and glycerin are provided. Stearic acid and lecithin and mono- and di-glycerides are particularly preferred.

Waxes such as beeswax and microcrystalline wax, fats/oils from sources such as soybeans and cottonseeds may be added as a part of the gum base formulation. These compounds also function as a softening agent for the gum base. Typically, these compounds either alone or in combination will comprise from 0 up to 25% of the gum base matrix. More preferably they comprise about 5 to 25% by weight of the gum base matrix. Specially desirable formulations will include a combination of microcrystalline wax and partially hydrogenated soybean oil in approximately 1:2 weight ratio.

A chewing formulation according to the present invention can contain a bulk sweetener selected from, but not limiting to sorbitol, xylitol, sucralose, cyclamate, glycyrrhizin, etc. Sorbitol and xylitol are particularly preferred either alone or in combination.

In addition to a sweetening agent the composition of this invention will contain one or more of the flavoring agents. This may include any of the industry's available and natural and synthetically derived food and pharmaceutical flavors. Specially preferred are those materials which impart a cooling and/or vaporizing sensation to the consumer upon mastication of gum for an improved mouth feel. As non-limiting examples, peppermint, spearmint, wintergreen, cinnamon and menthol are desirable. Typical flavoring and cooling agents will comprise 0 to 10% of the chewing gum composition, more preferably 0.1 to 0.5%.

LAE as the active ingredient may be provided in the form of an encapsulation (WO 2007/014580). Encapsulated LAE with flavor may provide for increased uniformity in the final formulation. Encapsulation may also impart a greater degree of stability to LAE and flavor during prolonged period of commercial storage. Encapsulating materials can also regulate the dissolution of LAE and flavor on a more sustained basis. Encapsulation may be accomplished by methods known in the art. Since chewing gum is considered to be a food product, food grade materials are desirable for the encapsulation. These materials include edible oleaginous substances (fats and oils) as well as saccharide protein and other non-toxic polymeric materials. Preferred materials for encapsulating are stearine as well as mono- and di-glyceride fat products such as canola, cottonseed and soybean oils.

Further compounds that may be combined with LAE in this type of application are surfactants with high HLB value (i.e.: hydrophil/lipophilic (hydrophobe) balance) such as polysorbates, the preferred one being sorbitan monolaurate.

### EXAMPLES

A formulation of chewing gum and a formulation of lozenge were used as examples of oral consumption products formulated with a cationic surfactant such as LAE. These compositions are the examples used to show the surprising effects of LAE against dental erosion.

### Chewing Gum:

| | Content |
|---|---|
| Formulation | (% by weight) |
| Gum base (natural or synthetic | 20-30% |
| elastomer filler, i.e. gum | |
| arabic and sorbitol) | |
| Sorbitol | 10-20% |
| LAE | 0.001 to 2% |
| Plasticizer | 0.1 to 1.5% |
| Flavoring | 0.1 to 2.5% |
| Sweetener | q.s. 100% |

### Lozenge:

| | Content |
|---|---|
| Formulation | (% by weight) |
| Humectant | 75 to 85% |
| Nonionic surfactant | 1-20% |
| LAE | 0.1 to 1.5% |
| Tableting lubricant | 0.1 to 5% |
| Sweetener | 0.2 to 2% |
| Sorbitol | 0.1 to 2.5% |

### Validation of Tooth Coating Effect by LAE

The tooth coating effect by LAE was validated by the methods described by Glantz PO (1981. “Adhesion in the oral cavity” In Fundamentals and application of surface interactions in the oral cavity (ed. S. A. Leach), pp 49-64. Information Retrieval Ltd. London). The method measures contact angles of sessile drops from liquids with a range of polarities to determine the polar component of the surface energy (gamma p) and dispersive component on teeth coated with LAE. Without LAE the surface energy was 23 dyn/cm. The surface energy reduction indicates that tooth surface is wetted (coated) by LAE. As shown in table 2, LAE at 0.0001% reduced surface energy indicating strong coating of tooth surfaces. In the presence of LAE the surface energy was reduced to a lowest level of 11.5 dyn/cm. This coating provides a shielding effect on teeth against erosion caused by acids from food and beverages.

Effect of LAE on Acids Generated from Sugar Rinses in Humans

It has been established, that beverages and food containing sugars promote tooth erosion. The American Dental Association (ADA) food and nutrition programs recommend a procedure for evaluating pH in the mouth after ingestion of food and beverages. This is summarized in the FDA guidelines published in Federal Register 43433, vol 61, No. 165, 1996. The procedure involved measuring pH on tooth plaque interfaces after giving the subjects (6 subjects per group) 10% sucrose rinse in the presence or absence of treatment with 0.16% LAE versus placebo solution containing flavor, sweetener, plasticizer (components of chewing gum) to assess their acid neutralizing effects. The data summarized in table 5 showed, that in the presence of the placebo, the pH of the tooth interface was 4.3±0.2 (danger zone is below pH 5) at 3 and 7 days of continuous monitoring. Whereas rinsing with LAE maintains a pH of 5.4±0.3 which is above the danger zone maintained for 3 and 7 days of continuous monitoring. This validates that LAE not only coats the surface of teeth but maintains a healthy pH around teeth to prevent erosion.

The efficacy of LAE solutions (rinse) or slurries containing LAE and other ingredients like flavors, surfactant and sweetener has been investigated.

In general, it has been observed that brief exposures of <3 min/day to oral care products results in fast re-growth of plaque bacteria.

### Model Studied:

The biofilm model is a reliable tool to predict the in vivo efficacy of antimicrobials, and de- and remineralization of enamel exposed to biofilms in vitro. The experiments were performed with the Zurich Biofilm Model. Biofilms are formed either on hydroxyapatite (HA) or bovine enamel disks that have preconditioned in pooled, unstimulated saliva. Biofilms are formed in 24-well cell culture dishes incubated anaerobically at 37° C. The detailed description to the preparation of this model was published by Guggenheim et al., 2001, J. Dent. Res. 80 (1) 363-370. The microbial composition of this biofilm was constituted by five bacterial species and a yeast. More specifically, they are as follows: Streptococcus oralis, Streptococcus sobrinus, Actinomyces naeslundii, Veillonella dispar and Fusobacterium nucleatum and the yeast studied was Candida albicans. The bacteria chosen for incorporation into this biofilm model were five species encountered in supragingival plaque.

Most of the biofilm models are based on a continuous flow culture system which means that biofilms are subjected to shear or detachment forces. In contrast, the biofilm model used in these experiments was based on a batch culture system that was subjected only intermittently to such forces in the course of dip-washing or gentle swirling. Thus, this biofilm model allows for controlled substance exposure times resembling those encountered during mouth rinsing.

The biofilms were exposed to LAE only 6 times during 1 minute in two subsequent days. The evaluation of the LAE effects was studied 16 h after the last exposure.

### Results:

### 1—The Reduction of the Surface Energy Produced by the Presence of LAE:

The surface energy of enamel is ca 23 dyn/cm. This surface energy is reduced by the presence of LAE to the following values:

TABLE 2
Surface energy versus LAE concentration

| | Surface Energy |
|---|---|
| [LAE] (%) | (dyn/cm) |
| 0.00001 | 21.5 |
| 0.0001 | 17 |
| 0.001 | 16.5 |
| 0.01 | 11.5 |
| 0.1 | 14 |
| 1 | 12 |

The reduction of the surface energy produced by the presence of LAE implies that the ability of microorganisms responsible of dental plaque formation to be adhered onto the teeth is considerably reduced.

2—Antimicrobial Activity of LAE on the Biofilm with Five Bacteria and the Yeast:

The results obtained with a control biofilm were compared with a biofilm treated with 0.5% LAE.

The results of the following table were expressed as log₁₀ of CFU:

TABLE 3
Antimicrobial activity of LAE versus control

| Microorganism | Control | 0.5% LAE |
|---|---|---|
| A. naeslundii | 8.3 | 2.5 |
| V. dispar | 8.1 | 5.0 |
| F. nucleatum | 5.1 | 2.1 |
| S. sobrinus | 8.6 | 6.1 |
| S. oralis | 9.0 | 6.1 |
| C. albicans | 5.1 | 2.1 |

The results demonstrate the efficacy of the antimicrobial effect of LAE in a biofilm.

3—Re-growth of biofilms after LAE exposure:

This experiment compared the re-growth of microorganisms in a control biofilm against a biofilm exposed to LAE. The content of microorganisms in each biofilm was studied along the time. For each point of analysis the growth of microorganisms was studied before and after a dip in a saline solution for control biofilms and in LAE for the other biofilms after each feeding.

The results are expressed as log₁₀ of CFU:

TABLE 4
Antimicrobial activity of LAE versus control

| Time of analysis | | Control | 1% LAE |
|---|---|---|---|
| 16.5 h | Before 1 dip | 7.1 | 7.1 |
| | After 1 dip | 7.2 | 3.5 |
| 24.5 h | Before 3 dip | 8.1 | 1.4 |
| | After 3 dip | 7.4 | 2.2 |
| 40.5 h | Before 4 dip | 8.1 | 5.9 |
| | After 4 dip | 8.1 | 1.2 |
| 48.5 h | Before 6 dip | 8.5 | 3.2 |
| | After 6 dip | 8.4 | 1.2 |
| 64.5 h | Endpoint | 9.0 | 4.3 |

### 4—Effect of LAE Regulating the pH:

Another effect produced by LAE is the pH neutralization. Upon hydrolysis LAE forms arginine which is a natural pH neutralizer in human saliva. The following table reports the intraoral pH with LAE versus a placebo from 0 to 7 days after 10% sucrose rinse:

TABLE 5
Effect of LAE regulating the pH

| Days | Placebo | LAE |
|---|---|---|
| 0 | 6 | 6 |
| 3 | 4.3 | 5.3 |
| 7 | 4.3 | 5.2 |

From these experiments, the following innovative points are observed:
LAE has a long-lasting protection in the oral cavity.
LAE acts as an anti-adherent agent of bacteria. That is, the presence of LAE in the oral cavity serves to resist the re-colonization of microorganisms responsible of teeth erosion. Thus, LAE has a role of anti-attachment of bacteria. This allows the reduction of the brushing with abrasive cleansers at the same time as it reduces the number of bacteria responsible to damage tooth and oral cavity.
LAE has a neutralising effect due to its hydrolysis to arginine and this maintains the pH balance.

The consumption of these products implies the release of LAE in the oral cavity and the protection of teeth from dental erosion and the oral cavity.

1-24. (canceled)

25. A method of protecting teeth against dental erosion comprising applying to teeth a composition including a cationic surfactant derived from the condensation of fatty acids and esterified dibasic amino acids, according to the following formula (1): 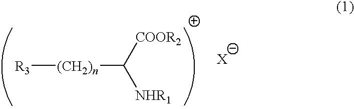
wherein
X⁻ is a counter ion derived from an organic or inorganic acid, selected from the group consisting of Br⁻, Cl⁻ or HSO₄⁻, or C₆H₅O⁻;
R₁ is a straight alkyl chain from a saturated fatty acid or hydroxyl acid having from 8 to 14 atoms linked to the α-amino acid group via an amidic bond;
R₂ is a straight or branched alkyl chain having from 1 to 18 carbon atoms or an aromatic group;
R₃: is 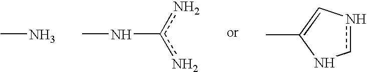
and n is from 0 to 4.

26. The method of claim 25, wherein the composition including a cationic surfactant is a composition for oral use selected from the group consisting of sweets, candies, tablets, lozenges, lollipops, chews, jellies, gums, drops or dry powder blends intended for dissolution to powdered drinks.

27. The method of claim 26, wherein the composition for oral use is a chewing gum or a lozenge.

28. The method of claim 25, wherein the cationic surfactant is present in the composition at a concentration from 0.001% to 5% by weight.

29. The method of claim 25 wherein applying to teeth a composition including a cationic surfactant comprises eating, chewing or sucking the composition.

30. The method of claim 25, wherein the cationic surfactant is the ethyl ester of the lauramide of arginine hydrochloride (LAE).

31. A composition for oral use, such as sweets, candies, tablets, lozenges, lollipops, chews, jellies, gums, drops, dry powder blends such as powdered drinks intended for dissolution, containing a cationic surfactant derived from the condensation of fatty acids and esterified dibasic amino acids, according to the following formula (1): 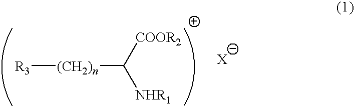
wherein
X⁻ is a counter ion derived from an organic or inorganic acid, selected from the group consisting of Br⁻, Cl⁻ or HSO₄⁻, or C₆H₅O⁻;
R₁ is a straight alkyl chain from a saturated fatty acid or hydroxyl acid having from 8 to 14 atoms linked to the α-amino acid group via an amidic bond;
R₂ is a straight or branched alkyl chain having from 1 to 18 carbon atoms or an aromatic group;
R₃: is 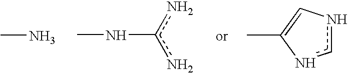
and n is from 0 to 4.

32. The composition of claim 31, wherein the composition further comprises a gum base material.

33. The composition of claim 31, wherein the composition further comprises at least one selected from the group consisting of chicle, jelutong, gutta percha, crown gum, butadiene-styrene copolymer, isobutylene and isoprene copolymer, polyethylene, polyisobutylene, polyester, and polyvinyl acetate.

34. The composition of claim 31, wherein the cationic surfactant is encapsulated in at least one polymeric coating.

35. The composition of claim 31, wherein the cationic surfactant is dispersed in a mass of water-swellable cellulosic powder or fibers forming a coherent network as a matrix.

36. The composition of claim 31, wherein the composition further comprises a gum base matrix containing ingredients selected from the group consisting of plasticizer, softener, lecithin, monoglycerides, diglycerides, lanolin, stearic acid, potassium stearate, glycerol triacetate and glycerin.

37. The composition of claim 31, wherein the composition further comprises at least one of (1) a wax added to a gum base matrix, the wax selected from the group consisting of beeswax and microcrystalline wax and (2) fats or oils added to a gum base matrix, the fats or oils selected from the group consisting of soybean oil or fat and cottonseed oil or fat.

38. The composition of claim 37, wherein the waxes, fats or oils comprise up to 50% by weight of gum base.

39. The composition of claim 31, wherein the composition further comprises a combination of microcrystalline wax and partially hydrogenated soya bean oil in a weight ratio of approximately 1:2.

40. The composition of claim 31, wherein the composition further comprises at least one sweetener selected from the group consisting of sorbitol, xylitol, sucralose, cyclamate and glycyrrhizin.

41. The composition of claim 31, wherein the composition further comprises flavoring and cooling agents in an amount of 0 to 10% by weight of the composition.

42. The composition of claim 31, wherein the composition further comprises at least one edible substance selected from the group consisting of oleaginous substances, fats, oils, saccharide proteins, stearines, monoglycerides, diglycerides, fat products, canola oil, cottonseed oil and soybean oil, wherein the at least one edible substance serves to encapsulate the cationic surfactant.

43. The composition of claim 31, wherein the composition is a chewing gum or a lozenge.

44. The composition of claim 31, wherein the cationic surfactant is present in an amount of 0.001 to 5% by weight of the composition.
